(19) United States
(12) Patent Application Publication
(10) Pub. No.: US 2023/0038770 A1
(43) Pub. Date: Feb. 9, 2023

(54) LIGHT DOSAGE ADMINISTERING DEVICE INCLUDING OPTICAL ASSEMBLY AND METHOD OF ADMINSTERING A DOSAGE OF LIGHT

(71) Applicant: INTO Technologies Inc., San Francisco, CA (US)

(72) Inventors: Justin CHEUNG, Pymble (AU); William MEEKER, San Francisco, CA (US)

(21) Appl. No.: 17/877,080

(22) Filed: Jul. 29, 2022

Related U.S. Application Data
(60) Provisional application No. 63/227,672, filed on Jul. 30, 2021.

(51) Int. Cl. A61N 5/06

(52) U.S. Cl. CPC A61N 5/0613; A61N 2005/0642; A61N 2005/0651; A61N 2005/0665

(57) ABSTRACT

Light administering device and methods of administering a dosage of light using the light administering device. The light administering device includes a light source configured to emit light rays and a collimating lens. The collimating lens is configured to collimate the light rays emitted from the light source such that the light rays are aligned in a light-emitting direction. The light administering device is configured to administering a dosage of light in the light-emitting direction towards the eyes of a user. The light administering device including may include plurality of light sources.

### CROSS-REFERENCE TO RELATED APPLICATION

This application claims the benefit under 35 U.S.C. § 119(e) of U.S. Provisional Patent Application No. 63/227,672, filed Jul. 30, 2021, and titled “LIGHT DOSAGE ADMINISTERING DEVICE INCLUDING OPTICAL ASSEMBLY,” the entirety of which is incorporated herein by reference.

### FIELD OF THE INVENTION

The invention relates to a light dosage administering device including an optical assembly for directing light emitted by a light source in the light dosage administering device, and methods for administering a dosage light using the light dosage administering device.

### BACKGROUND OF THE INVENTION

Pharmaceuticals are a customary solution to treating numerous medical indications. It has been proposed that light can be used to treat these medical indications as an alternative or a supplement to pharmaceuticals.

### SUMMARY OF THE INVENTION

In one aspect, the invention relates to a light administering device capable of administering a dosage of light in a light-emitting direction towards the eyes of a user. The light administering device includes a light emitter including a plurality of light sources and a collimating lens. The plurality of light sources including a plurality of first light sources configured to emit light rays of a first wavelength, a plurality of second light sources configured to emit light rays of a second wavelength, and a plurality of third light sources configured to emit light rays of a third wavelength. The second wavelength is different from the first wavelength, and the third wavelength is different from the first wavelength and the second wavelength. The collimating lens is configured to collimate the emitted light rays from each light source of the light emitter such that light rays emitted from the light emitter and collimated by the collimating lens are aligned in the light-emitting direction.

In another aspect, the invention relates to a method of administering a dosage of light including placing a light administering device in front of the eyes of a patient, emitting light rays from at least one light source of the light administering device, collimating the light rays emitted from the least one light source such that the light rays are aligned in a light-emitting direction, and projecting the collimated light rays onto a target area. The light-emitting direction is a direction towards the eyes of a patient. The target area includes the eyes of the patient. The collimated light rays are projected onto the target area while the eyes of the patient are closed.

In a further aspect, the invention relates to a light administering device capable of administering a dosage of light in a light-emitting direction towards the eyes of a user. The light administering device includes a plurality of light sources and a collimating lens. The plurality of light sources is arranged in an array of rows and columns. The array of light sources having at least three columns of light sources and at least three rows of light sources and more columns of light sources than rows of light sources. Each light source of the plurality of light sources is configured to emit light rays. The collimating lens is configured to collimate the emitted light rays from each light source of the plurality of light sources such that the light rays emitted from the plurality of light sources and collimated by the collimating lens are aligned in the light-emitting direction.

In still another aspect, the invention relates to a light administering device capable of administering a dosage of light in a light-emitting direction towards the eyes of a user. The light administering device includes a light source configured to emit light rays, a collimating lens, and at least one optical diffuser. The collimating lens is configured to collimate the light rays emitted from the light source such that the light rays are aligned in the light-emitting direction. The at least one optical diffuser receives collimated light rays transmitted through the collimating lens and is configured to diffuse the collimated light rays.

In yet another aspect, the invention relates to a light administering device. The light administering device includes a light source configured to emit light rays and a collimating lens. The collimating lens is configured to collimate the light rays emitted from the light source such that the light rays are aligned in a light-emitting direction. The light administering device is configured to administer a dosage of light in the light-emitting direction towards the eyes of a user.

These and other aspects of the invention will become apparent from the following disclosure.

### BRIEF DESCRIPTION OF THE DRAWINGS

FIGS. 1A and 1B show a light dosage administering device according to an embodiment of the invention. FIG. 1A is a perspective view showing the front and top sides of the light dosage administering device and FIG. 1B is a perspective view showing the back and bottom sides of the light dosage administering device.

FIG. 2 shows the light dosage administering device being held in front of a user's eyes by hand.

FIG. 3 shows the light dosage administering device being held in front of a user's eyes by a stand.

FIG. 4 is an exploded view of the light dosage administering device.

FIG. 5 is a top view of an LED board of the light dosage administering device.

FIG. 6 is a schematic, cross-sectional view of an optical assembly for a single light emitting diode (light source) of the light dosage administering device.

FIG. 7 is a schematic, cross-sectional view of the optical assembly for a plurality of light emitting diodes (light sources).

FIG. 8 is a top view of the collimating lens of the optical assembly.

FIG. 9 is a top view of both the collimating lens and the LED board.

FIG. 10 is a schematic, cross-sectional view of a variation of the optical assembly for a plurality of light emitting diodes (light sources).

FIG. 11 is a detail view of the light emitting diode and a total internal reflection (TIR) collimating lens of the optical assembly shown in FIG. 6.

FIG. 12 is a schematic, cross-sectional view of an alternative optical assembly for a single light emitting diode (light source) of the light dosage administering device.

### DETAILED DESCRIPTION OF THE PREFERRED EMBODIMENTS

Medical indications may be treated by light. These medical indications include not only physical and/or physiological ailments, but also human functions (e.g., cognitive abilities and alertness). Such medical indications include, for example, insomnia relief, mitigation of stress and anxiety, relaxation and tension relief, and headache and migraine reduction. These medical indications may be treated by administering one or more dosages of light to a user (or patient). The dosages of light may be administered to the eyes of the user to stimulate the retinal ganglion cells within the eyes of a user. The dosages of light may be administered while the eyes are closed, with the light being transmitted through eyelids of the user. The dosages of light may be administered according to various parameters including wavelength, area within the user's field of vision, intensity, pulse frequency, duration, pulse waveform shape, photon quantity, or any combination thereof. Additional details of treating such medical indications are described in U.S. Provisional Patent Application No. 63/171,900, filed Apr. 7, 2021, and U.S. patent application Ser. No. 17/714,756, filed Apr. 6, 2022, the disclosures of which are hereby incorporated by reference in their entirety.

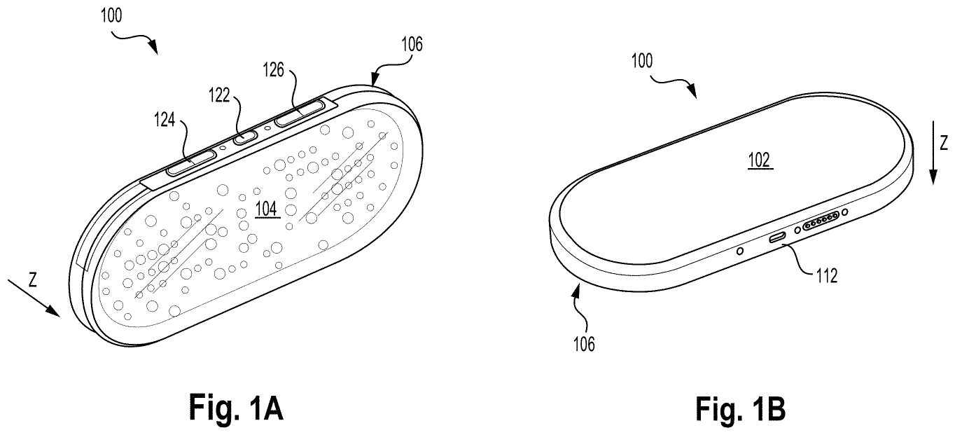

One aspect of the present invention relates to a light dosage administering device that is employed to administer dosages of light to the user. FIGS. 1A and 1B show a light dosage administering device 100 according to an embodiment of the invention. FIG. 1A is a perspective view showing the front and top sides of the light dosage administering device 100, while FIG. 1B is a perspective view showing the back and bottom sides of the light dosage administering device 100. The light dosage administering device 100 includes a front surface 104, a back surface 102, and a perimeter surface 106. The front surface 104 may be transparent so as to allow light generated within the light dosage administering device 100 to be emitted therethrough along direction Z. The back surface 102 may be opaque so as to block light passing therethrough.

The light dosage administering device 100 may include various inputs, outputs, and/or user controls. For example, in one embodiment, the light dosage administering device 100 may include a USB port 112 (e.g., USB-micro or USB-C), a power button 122, and rocker switches 124, 126. In one embodiment, the light dosage administering device 100 may include one or more status display indicators (e.g., single or multi-color LED emitters, matrix LCD or LED panel for displaying alphanumeric symbols/text, etc.) for conveying the current operating status of the device. In one embodiment, the light dosage administering device 100 may include one or more touch-sensitive panels or touch screens. Of course, it will be appreciated that the light dosage administering device 100 may employ any combination of the aforementioned features and/or any other user input/output interface features without departing from the spirit of the invention.

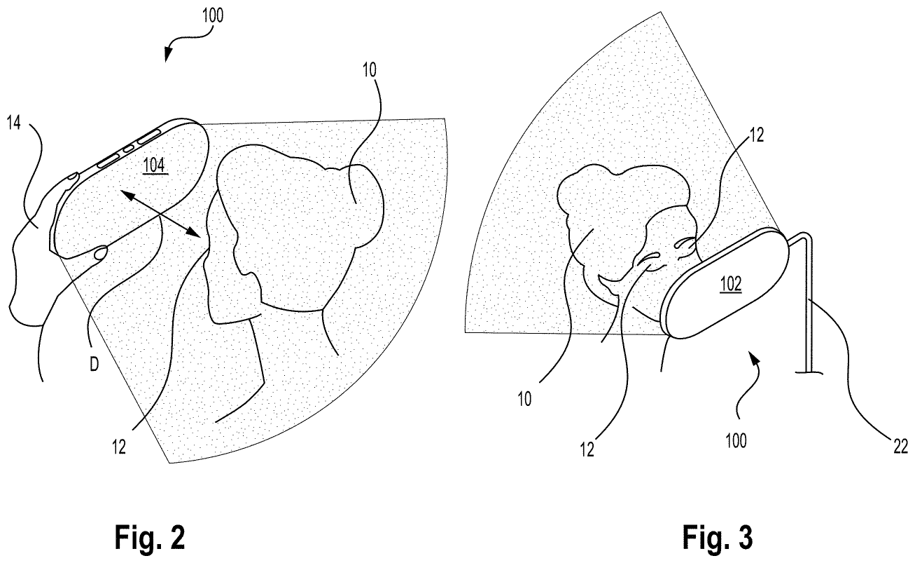

FIGS. 2 and 3 illustrate exemplary operations in connection with the light dosage administering device 100. The light dosage administering device 100 is held in front of the eyes 12 of a user 10, as shown in FIGS. 2 and 3, to administer the dosages of light. In one embodiment as illustrated in FIG. 2, a user 10 may hold the light dosage administering device 100 in front of his/her eyes 12 using his/her hand 14 at a distance D from his/her eyes 12. As the user 10 holds the light dosage administering device 100 in front of his/her eyes 12, the user 10 may operate the device, and the light dosage administering device 100 emits light from the front surface 104 to provide the dosages of light. The front surface 104 of this embodiment is an eye-facing surface of the light dosage administering device 100. In this embodiment, light is emitted from nearly the entirety of the front surface 104. But, the design is not so limited, and light may be emitted from a smaller portion of the front surface 104 or other portions of the light dosage administering device 100.

In one embodiment, the light dosage administering device 100 is arranged in size and profile to be comfortably held in the hand 14 of the user 10 without undue fatigue. Preferably, the components of the light dosage administering device 100 are designed with such uses in mind and are designed to be small and lightweight.

In one embodiment as illustrated in FIG. 3, the light dosage administering device 100 may be coupled to a stand 22 during the administration of light dosages, thereby avoiding the need for the user to physically hold the device during its operation.

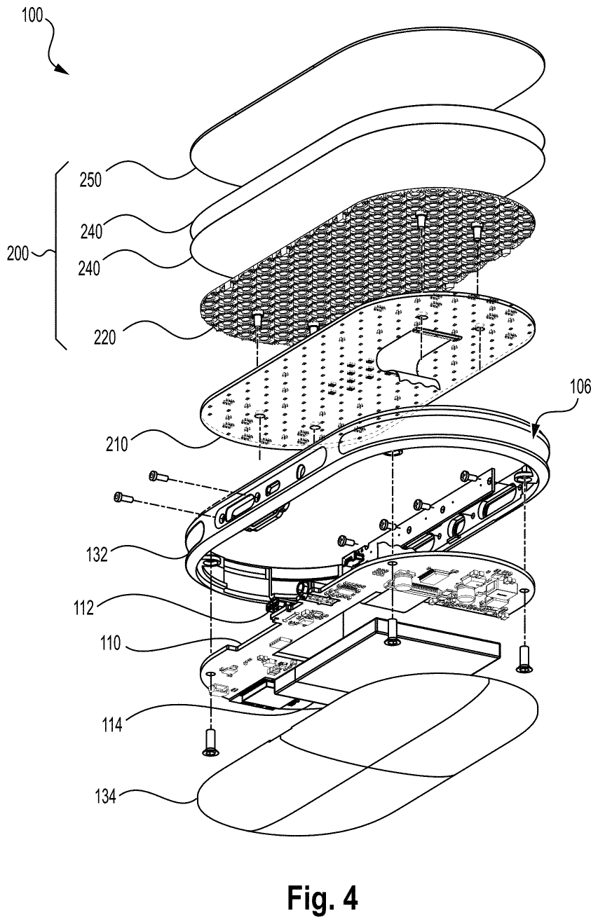

FIG. 4 is an exploded view of the light dosage administering device 100 according to one embodiment. The light dosage administering device 100 may include a frame 132 forming the perimeter surface 106 of the device, a back cover 134 forming the back surface 102 of the device, and a transparent cover 250 forming the front surface 104 of the device. Each of the frame 132 and the back cover 134 may be made from any suitable material, including, for example, aluminum or a resin material. As will be discussed further below, the light emitted from the light emitting diodes 212 is emitted from the light dosage administering device 100 through the transparent cover 250. In one embodiment, the transparent cover 250 may be a separate component from the frame 132, and the frame 132 may be arranged to receive the transparent cover 250. In one embodiment, the back cover 134 may be a separate component from the frame 132, and the frame 132 may be arranged to receive the back cover 134. In one embodiment, the transparent cover 250 and/or the back cover 134 may be integral with the frame 132.

The light dosage administering device 100 may also include a control board 110 and an LED board 210. The control board 110 may be used to control the operation of the light dosage administering device 100, and more specifically, may be used to selectively control light emitting diodes on the LED board 210. In the embodiment shown in FIG. 4, the control board 110 and the LED board 210 are shown as separate components electrically, communicatively, and operatively connected to each other, but on other embodiments, the control board 110 and the LED board 210 may be integrally formed as, for example, a printed circuit board. The control board 110 may be connected (e.g., electrically) to the LED board 210 by a suitable connection such as wires and/or a ZIF socket and corresponding pins.

The control board 110 may be a microprocessor-based control board having one or more processors and one or more memories. The processor can be any suitable processing device, including, but not limited to, a microprocessor, a microcontroller, an integrated circuit, a logic device, a programmable logic controller (PLC), an application specific integrated circuit (ASIC), and/or a Field Programmable Gate Array (FPGA). The memory can include one or more computer-readable media, including, but not limited to, non-transitory computer-readable media, a computer readable-non-volatile medium (e.g., a flash memory), a RAM, a ROM, hard drives, flash drives, and/or other memory devices. The memory can store information accessible by the processor, including computer-readable instructions that can be executed by the processor. The instructions can be any set of instructions or a sequence of instructions that, when executed by the processor, cause the processor to perform operations. In some embodiments, the instructions can be executed by the processor to cause the processor to complete any of the operations and functions for which the light dosage administering device 100 is configured, such as those discussed herein. The instructions can be software written in any suitable programming language or can be implemented in hardware. Additionally, and/or alternatively, the instructions can be executed in logically and/or virtually separate threads on the processor. The memory can further store data that can be accessed by the processor.

The control board 110 may be coupled to the power button 122 and rocker switches 124, 126, shown in FIG. 1A, along with any/all of the aforementioned user interface components. Such user interface components are coupled to the control board 110 and processor to receive input from the user 10 and/or provide information output to the user 10. The control board 110 may thus be configured to receive input from these user interfaces to change the operation of the light dosage administering device 100 and selectively control the light emitting diodes 212. Other devices, such as personal computers for example, may be communicatively coupled to the control board 110 and the processor to control or otherwise program the light dosage administering device 100. Any suitable connection may be used to communicatively couple the other device to the light dosage administering device 100. Suitable connections include, for example, an electrical conductor; a low-level serial data connection, such as Recommended Standard (RS) 232 or RS-485; a high-level serial data connection, such as Universal Serial Bus (USB) or the Institute of Electrical and Electronics Engineers (IEEE) 1394; a parallel data connection, such as IEEE 1284 or IEEE 488; and/or a short-range wireless communication channel, such as BLUETOOTH®; and/or wireless communication networks using radio frequency signals, such as WiFi®. When a wireless protocol is used, each of the other device and the light dosage administering device 100 may include a transmitter and a receiver. In this embodiment for example, USB may be used, and the light dosage administering device 100, more specifically the control board 110, may include (or be separately coupled to) a USB (e.g., USB-micro, USB-C) port 112, as shown in FIGS. 1A and 4.

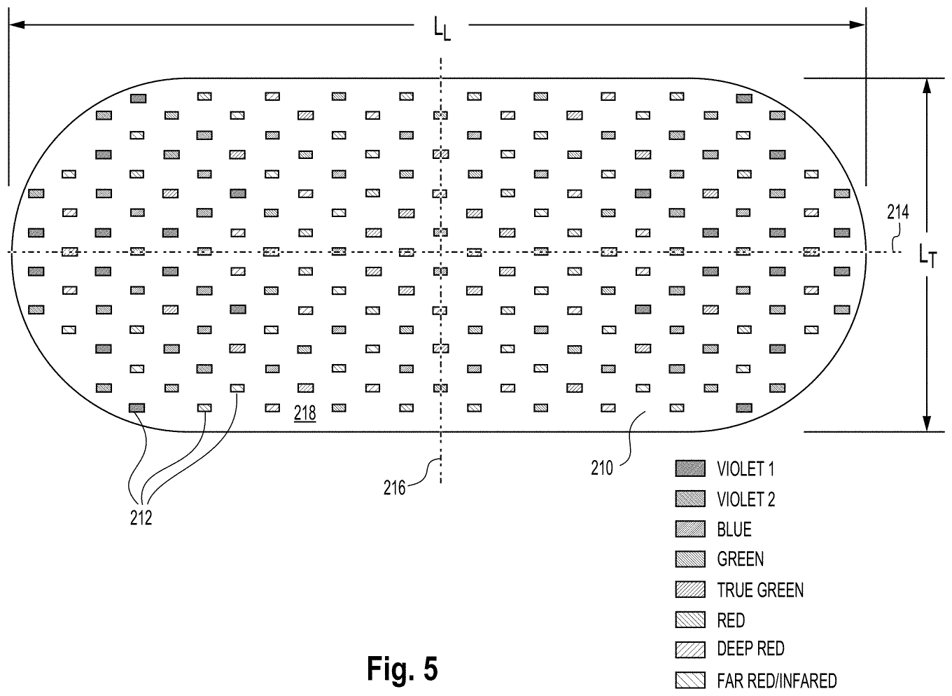

FIG. 5 is a top view of the LED board 210. The LED board 210 may include a plurality of light emitting diodes 212. The LED board 210 may be a printed circuit board, and the light emitting diodes 212 may be attached to an outer surface 218 of the LED board 210 by any suitable means such as by soldering. For clarity, only some of the light emitting diodes 212 are labeled with a reference numeral in FIG. 5. The light emitting diodes 212 are arrayed in two directions on the LED board 210. For convenience, the horizontal and vertical directions in the view of FIG. 5 will be referred to as the longitudinal and transverse directions, respectively, of the array of light emitting diodes 212 and the light dosage administering device 100 overall. The LED board 210, and thus the array of light emitting diodes 212, has a length in the longitudinal direction (longitudinal length LL) and a length in the transverse direction (transverse length LT). As noted above, light is emitted from nearly the entirety of the front surface 104 of the light dosage administering device 100, and thus, the longitudinal and transverse lengths LT and LL of the array of light emitting diodes 212 may be nearly the same as those same dimensions of the overall device. As the light dosage administering device 100 is preferably sized and shaped to fit comfortably in the hand 14 of the user 10, the longitudinal length LL preferably may be 17 cm and the transverse length LT preferably may be 7 cm. Such a size is also suitable to administer the dosage of light to the eyes 12 of the user 10 and illuminate the entire eye regions at distances D of 5 cm (about 2 inches) to 40 cm (about 16 inches) from the eyes 12 (or face) of the user 10 (see FIG. 2).

As shown in FIG. 4, the light dosage administering device 100 may additionally include a collimating lens 220 and at least one diffusion film 240. In one embodiment, the device may include multiple diffusion films. For example, as illustrated in FIG. 4, the device may include two diffusion films. The collimating lens 220, diffusion film(s) 240, and transparent cover 250 together form an optical assembly 200.

The light dosage administering device 100 may include a power source to power the components therein, especially the control board 110 and the LED board 210 and its light emitting diodes 212. As the light dosage administering device 100 is preferably a handheld device in one embodiment, the device may include a battery 114, as shown in FIG. 4. Any suitable rechargeable or non-rechargable battery 114 may be used. In one embodiment, the battery 114 is a rechargeable lithium polymer battery. In the case of the battery 114 being rechargeable, the battery 114 may be charged through a charging port, such as the USB (e.g., USB-micro or USB-C) port 112.

As the light dosage administering device 100 is configured to administer dosages of light for treatment of medical indications, the components of the light dosage administering device 100 are also designed with the quality of light administered to the user 10 in mind. The light dosage administering device 100 may be designed to provide an experience that is effective in treating such medical indications and such features are discussed below.

The light dosage administering device 100 includes at least one light source that generates the light of specified dosages to be administered to the user 10. In one embodiment, the light source is a light emitting diode, and in one embodiment, the light source is a plurality of light emitting diodes 212 (see FIG. 5) disposed on the LED board 210. Although any suitable light source may be used, such as incandescent light bulbs, advantages of using light emitting diodes 212 include their small size, low power consumption, and precision in controlling the amount of emitted light. In addition, wavelengths of the light administered to the user 10 are preferably controlled depending upon the treatment, and light emitting diodes 212 are able to produce a wide range of wavelengths of light. The light emitting diodes 212 may emit specific wavelengths of light and may be selected in order to provide specified wavelengths in a particular arrangement to the user 10. In some embodiments, the light emitting diodes 212 may be light emitting diodes 212 in which the wavelength of light can be adjusted or changed, such as an RGB light emitting diode. Although the light emitting diodes 212 discussed herein are semiconductor-based light emitting diodes 212, other light emitting diodes 212 may be used including organic light emitting diodes. One or more of the light emitting diodes 212 may be micro LEDs.

In one embodiment as depicted in FIG. 1A, the front surface 104 of the light dosage administering device 100, through which light is emitted, has an oblong shape. In one embodiment, the front surface 104 is generally rectangular having semicircular end portions. The oblong shape may be preferred to both comfortably fit in the hand 14 of the user 10 and to provide the desired illumination profile. Other suitable shapes may be used, including other oblong shapes such as, for example, rectangles having rounded or chamfered corners and ovals.

As shown in FIG. 5, the LED board 210, and thus the array of light emitting diodes 212, has a shape that corresponds to the shape of the front surface 104 and, in one embodiment, is the same as the shape of the front surface 104. The array of light emitting diodes 212 has a longitudinal centerline 214 extending in the longitudinal direction and a transverse centerline 216 extending in the transverse direction. In this embodiment, the array of light emitting diodes 212 is preferably symmetrical about each of the longitudinal centerline 214 and the transverse centerline 216. Also in this embodiment, the longitudinal centerline 214 and the transverse centerline 216 of the array of light emitting diodes 212 are also longitudinal and transverse centerlines for the light dosage administering device 100.

To provide the appropriate dosage of light to the user 10, the array of light emitting diodes 212 preferably includes at least three columns (in the transverse direction) and at least three rows (in the longitudinal direction) of light emitting diodes 212. More preferably, the array of light emitting diodes 212 includes at least four columns and at least four rows of light emitting diodes 212. In an embodiment shown in FIG. 5, the array of light emitting diodes 212 includes twenty-five columns of light emitting diodes 212, and, in the center of the array eight or nine rows of light emitting diodes 212. For clarity, the longitudinal direction may also be referred to as a column direction and the transverse direction may also be referred to as a row direction.

In this embodiment, the light emitting diodes 212 in adjacent columns are staggered. Various suitable staggering patterns may be used, but in this embodiment, the light emitting diodes 212 in one column are offset in the transverse direction from those in an adjacent column by half of the distance between successive light emitting diodes 212. For example, the light emitting diodes 212 in the next column are offset in the transverse direction from those in the previous column by half of the distance between successive light emitting diodes 212 in a column. The number of light emitting diodes 212 per column may decrease for the columns further away from the transverse centerline 216. That is, the columns towards the ends of the LED board 210 (array of light emitting diodes 212) in the longitudinal direction may contain fewer light emitting diodes 212 than those columns near the center of the LED board 210. The reduced numbers of light emitting diodes 212 in certain columns may be a result of the semicircular portions of the LED board 210. For example, in one embodiment, each end column along the length direction contains four light emitting diodes 212, while each of the next columns moving towards the center along the length direction contains five light emitting diodes 212.

As noted above, a variety of different light emitting diodes 212 may be used. In one embodiment, the light emitting diodes 212 include a plurality of different types, where each type is configured to emit a different wavelength of light. In one embodiment, the array of light emitting diodes 212 may include single wavelength light emitting diodes emitting a single wavelength (or at least a narrow wavelength band). Suitable single wavelength light emitting diodes and their corresponding wavelengths are shown in Table 1 below.

TABLE

| | | Viewing | Luminous | | |
|---|---|---|---|---|---|
| Wavelength | Nominal | Angle | Intensity | | Exemplary |
| (nm) | Color | (deg) | (mcd) | Exemplary Model | Manufacturer |
| 405 | Violet 1 | 130 | — | SM0603UV-405 | Bivar |
| 415 | Violet 2 | 150 | — | ATS2012UV415 | Kingbright |
| 468 | Blue | 130 | 115 | SM0603UBWC | Bivar |
| 515 | Green | 140 | 430 | 150060GS75000 | Wurth |
| 530 | True | 120 | 350 | IN-S63AT5G | Inolux |
| | Green | | | | |
| 639 | Amber | 130 | 54 | LTST-C191KRKT | Lite-On |
| 660 | Red | 140 | 16 | SML-310LTT86 | Rohm |
| 700 | Infrared | 140 | 5 | SML-LX15HC-RP-TR | Rohm |

In this embodiment, the array of light emitting diodes 212 includes at least one light emitting diode 212, and preferably more than one light emitting diode 212, having each of the wavelengths listed in Table 1. As such, in some embodiments, the array of light emitting diodes 212 will have discrete wavelengths from across the visual spectrum from near ultraviolet light to near infrared light, as opposed to a narrow band of wavelengths in the visual spectrum or white light (such as daylight or tungsten balanced light). Preferably, the array of light emitting diodes 212 will include at least three different types of single wavelength light emitting diodes 212, each having a different wavelength. For example, the array of light emitting diodes 212 may include a plurality of first light emitting diodes 212 configured to emit light of a first wavelength, a plurality of second light emitting diodes 212 configured to emit light of a second wavelength, and a plurality of third light emitting diodes 212 configured to emit light of a third wavelength. The second wavelength is different from the first wavelength, and the third wavelength is different from the first wavelength and the second wavelength. As such, the light emitting diodes 212 of this embodiment, being configured to emit discrete wavelengths, therefore do not emit a narrow band of wavelengths in the visual spectrum and do not emit white light (regardless of color temperature or tone, e.g., tungsten balanced light).

In another embodiment, the array of light emitting diodes 212 may include at least seven different types of single wavelength light emitting diodes 212, each having a different wavelength. The array of light emitting diodes 212 may include, for example, a plurality of first light emitting diodes 212 configured to emit light of a first wavelength, a plurality of second light emitting diodes 212 configured to emit light of a second wavelength, a plurality of third light emitting diodes 212 configured to emit light of a third wavelength, a plurality of fourth light emitting diodes 212 configured to emit light of a fourth wavelength, a plurality of fifth light emitting diodes 212 configured to emit light of a fifth wavelength, a plurality of sixth light emitting diodes 212 configured to emit light of a sixth wavelength, and a plurality of seventh light emitting diodes 212 configured to emit light of a seventh wavelength. Each of the first, second, third, fourth, fifth, sixth, and seventh wavelengths may be different from each other.

In a further embodiment, the array of light emitting diodes 212 may include at least eight different types of single wavelength light emitting diodes 212, each having a different wavelength. FIG. 5 is an example of such an embodiment that includes a plurality of first light emitting diodes 212 configured to emit light of a first wavelength, a plurality of second light emitting diodes 212 configured to emit light of a second wavelength, a plurality of third light emitting diodes 212 configured to emit light of a third wavelength, a plurality of fourth light emitting diodes 212 configured to emit light of a fourth wavelength, a plurality of fifth light emitting diodes 212 configured to emit light of a fifth wavelength, a plurality of sixth light emitting diodes 212 configured to emit light of a sixth wavelength, a plurality of seventh light emitting diodes 212 configured to emit light of a seventh wavelength, and a plurality of eighth light emitting diodes 212 configured to emit light of an eighth wavelength. Each of the first, second, third, fourth, fifth, sixth, seventh, and eight wavelengths may be different from each other.

Other suitable embodiments may similarly include four, five, or six different types of single wavelength light emitting diodes 212, each having a different wavelength, or a number of single wavelengths greater than eight.

FIG. 5 shows an example of a suitable arrangement for the different single wavelength light emitting diodes 212 on the LED board 210. The light emitting diodes 212 are arranged to provide the capability to deliver light dosages of specific wavelengths to specific areas of a user's field of vision. Such an arrangement may also provide a greater concentration of light emitters of particular wavelengths in certain regions. As shown in FIG. 5, for example, the light emitting diodes 212 having wavelengths of violet and/or ultraviolet light are concentrated at a peripheral region of the array and LED board 210, while light emitting diodes 212 having wavelengths of red and/or infrared light are concentrated in a central region of the array. For example, the average wavelength for the plurality of light emitting diodes 212 in the center third of the LED board 210, as split along the longitudinal direction of the LED board 210, is higher than the average wavelength for the plurality of light emitting diodes 212 in each of the outer third of the LED board 210, as split along the longitudinal direction of the LED board 210. The peripheral regions in the longitudinal direction correspond to the peripheral field/zone of vision of the user 10 as compared to central regions corresponding to the user's central field/zone of vision. The light emitting diodes 212 emitting ultraviolet and/or purple light wavelengths may, for example, be provided only at regions corresponding to a peripheral field/zone of vision of the user and not provided at regions corresponding to a central field/zone of vision. The light emitting diodes 212 emitting red and/or infrared light wavelengths may, for example, be provided only at regions corresponding to a central field/zone of vision of the user 10 and not provided at regions corresponding to a peripheral field/zone of vision.

Preferably the light dosage administering device 100 provides dynamic visual experiences for the user and can be used to administer dosages of light with different wavelengths based on the desired experiences. These experiences may include, for example, dosages of light produced with deep violet wavelengths for immersive natural feelings, dosages of light produced with blue wavelengths for an energizing experience, dosages of light produced with green wavelengths for a calming experience, dosages of light produced with red wavelengths to imitate dawn or dusk, and dosages of light produced with infrared wavelengths to improve acuity. In some embodiments, the acuity experience may be performed using the dosages of light with red wavelengths.

Such experiences are preferably produced using the array of the light emitting diodes 212 shown in FIG. 5, but other arrangements and smaller arrays may be used to produce such experiences. The light dosage administering device 100 preferably includes five different experiences and a light emitting diode 212 with a wavelength in the desired range for each experience. In some embodiments, the light dosage administering device 100 thus includes at least four, and more preferably at least five, different wavelengths of light emitting diodes 212. Preferably, one light emitting diode 212 of each of the different wavelengths is in each of four quadrants defined by the longitudinal centerline 214 and the transverse centerline 216. Accordingly, in some embodiments the array of light emitting diodes 212 preferably includes at least 16 light emitting diodes 212 and more preferably at least 20 light emitting diodes 212.

In these embodiments, various different arrays can be constructed provided at least one a light emitting diodes 212 of each different wavelength is in each quadrant. The light emitting diodes 212 in such arrays are preferably distributed to be mirrored across the transverse centerline 216. Across the longitudinal centerline 214, the distribution may be a mirror image in location, but it is not so limited, although the density of the light emitting diodes 212 will preferably match across the longitudinal centerline 214.

The array of light emitting diodes 212 may also be distributed to have a density over a given area. Preferably, the light emitting diodes 212 are distributed on the LED board 210 to have a density from one LED per 80 mm² to one LED per 25 mm², and more preferably from one LED per 60 mm² to one LED per 25 mm². Although the density may be uniform, the LEDs could be positioned to have a non-uniform density, for example, the density could be higher directly in front of the eyes (the central region) and lower in the peripheral regions.

The light dosage administering device 100 may also include one or more RGB light emitting diodes. The RGB light emitting diodes may be addressable RGB light emitting diodes such that the wavelength of each RGB light emitting diode can be changed independently of the other RGB light emitting diodes. A suitable RGB light emitting diode is Model No. SMLVN6RGB7W1 manufactured by ROHM Semiconductor. Although RGB light emitting diodes may be used in the array of light emitting diodes 212, the RGB light emitting diodes are used and positioned as status indicators, rather than to produce the dosage of light in this embodiment.

The light emitting diodes 212 in the array of light emitting diodes 212 may be selectively controlled, such as by controlling which light emitting diodes 212 are emitting light and/or the intensity of the light emitted from a light emitting diode 212 in the array. Additionally, when RGB light emitting diodes are used, the wavelength of light from the light emitting diode 212 may be selectively controlled. Such aspects may be controlled according to a predetermined pattern and/or duration to treat the medical indication as discussed above.

The light dosage administering device 100 may be provided with the optical assembly 200 to optimize the light emitted from the LED board 210, so as to provide a desired amount and profile of light for administration to the user. Such an optical assembly 200 may be preferred when light emitting diodes 212 are used as the light source, to avoid light emitted from the light emitting diodes 212 being perceived by the user as discrete points, which may detract from the user experience and may in some cases, influence efficacy of the treatment of the medical indication. The optical assembly 200 may also be referred to as an optical stack.

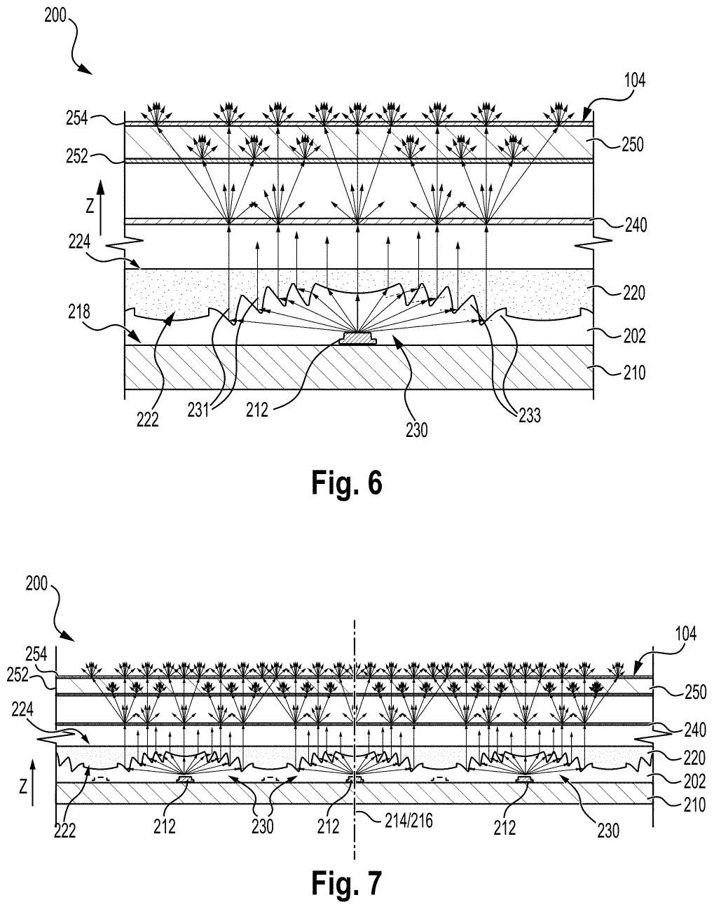

The optical assembly 200 will be further described with reference to FIGS. 6 and 7. FIG. 6 is a schematic, cross-sectional view of the optical assembly 200 for a single light emitting diode 212 (light source), and FIG. 7 is a schematic, cross-sectional view of the optical assembly 200 for a plurality of light emitting diodes 212 (light sources). To further the explanation of the optical assembly 200, it is noted that FIGS. 6 and 7 are not drawn to scale. The front surface 104 is a planar surface and has a normal direction that is outward away from the light dosage administering device 100 and towards the eyes 12 of the user. In the following discussion, this direction (again denoted as direction Z) is referred to as an outward direction or an eye facing direction, and the direction opposite to the outward direction is an inward direction.

When current passes through the light emitting diode 212, the light emitting diode 212 emits light. The emitted light has a wavelength and is emitted in rays of light. Only some exemplary rays of light from the light emitting diode 212 are shown in FIGS. 6 and 7. While the rays of light emitted from the light emitting diodes 212 travel in the outward direction, many of these rays also have significant components of the direction of travel that are in the longitudinal and/or transverse directions of the device. Absent optical adjustment via the optical assembly 200, the light with significant components in the longitudinal direction and/or transverse direction may not be directed towards the eyes 12 of the user 10 and instead may escape or be lost to the environment. In one embodiment, one or more (or all) of the light emitting diodes 212 has an emission spread angle of 130 degrees. In one embodiment, one or more (or all) of the light emitting diodes 212 has an emission spread angle of 120 degrees. In one embodiment, one or more (or all) of the light emitting diodes 212 has an emission spread angle of 140 degrees. In one embodiment, one or more (or all) of the light emitting diodes 212 has an emission spread angle of 150 degrees. In one embodiment, all of the light emitting diodes 212 have the same emission spread angle. In one embodiment, at least some of the light emitting diodes 212 have emission spread angles different from one another. In one embodiment, at least some of the light emitting diodes 212 have emission spread angles different from one another, and the emission spread angles of the light emitting diodes 212 have emission spread angles ranging from 120 to 150 degrees.

The collimating lens 220 of the optical assembly 200 is configured to collimate the rays emitted from each light emitting diode 212. In one embodiment, the collimating lens 220 includes a Fresnel surface to collimate the rays of light. For example, the collimating lens 220 may include an inner surface 222 facing the LED board 210 an outer surface 224 facing outward. The inner surface 222 may include the Fresnel surface formed thereon. In one embodiment, the Fresnel surface includes a plurality of Fresnel features 230, each Fresnel feature corresponding to a respective light emitting diode 212 of the LED board 210. In one embodiment, the collimating lens 220 includes a Fresnel feature 230 for each light emitting diode 212 of the LED board 210.

By collimating the rays of emitted light from the light emitting diodes 212 using the collimating lens 220, the rays become substantially parallel to each other in the outward/eye facing direction, which increases the efficiency of the light directed towards a target plane, such as the eyes 12 of the user 10.

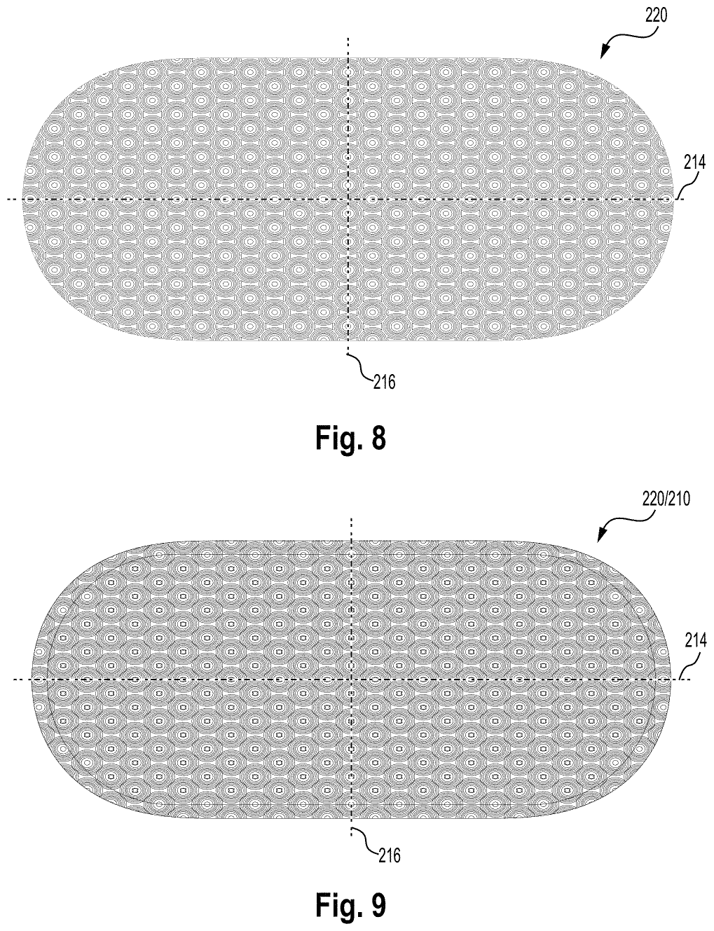

In one embodiment, each Fresnel feature 230 includes a plurality of concentric prisms 231. The plurality of concentric prisms 231 are separated from each other by a plurality of concentric valleys 233. FIG. 8 is a top view of the collimating lens 220, showing the concentric prisms 231. In one embodiment as depicted in FIG. 8, the concentric prisms 231 have a circular profile. However, it will be appreciated that other profiles may be used including, for example, an elliptical profile with the major axis in the transverse direction of the light dosage administering device 100 and the minor axis in the longitudinal direction. The target area for the dosage of light is the eyes 12 of a user 10. An elliptical profile may be preferred as such a profile helps create an elliptical projection more closely matching the target (eye 12), as compared to a circular projection that may project onto the entire face.

In one embodiment, each Fresnel feature 230 is aligned with and centered on the corresponding light emitting diode 212, and a top view of such a stack is shown in FIG. 9. However, it may be desirable to direct the light produced by those light emitting diodes 212 positioned further away from the longitudinal centerline 214 and the transverse centerline 216, towards these centerlines. Directing the light towards these centerlines may increase the efficiency of the light dosage administering device 100 and direct more overall light towards the eyes 12 of the user 10. In one embodiment, some of the Fresnel features 230 have their center axis offset from the center of the corresponding light emitting diode 212. In one example, this offset is a linear function of the distance that a light emitting diode 212 is from the center of the array (where the longitudinal centerline 214 and longitudinal centerline 214 intersect) and/or either one or both of the longitudinal centerline 214 or the transverse centerline 216. The offset may be applied to all of the light emitting diodes 212. The position may be set using, for example, equation (1):

$${X}_{lens} = {X}_{LED} ( 1 - \frac{1}{{c}_{offset}} ) \qquad ( 1 )$$

where Xₗₑₙₛ is the distance from the center of the array to the center of the Fresnel feature 230, X_{LED} is the distance from the center of the array to the center of the light emitting diode 212, and c_{offset} is a constant. The constant (c_{offset}) could be defined by the vertical distance between the light emitting diode 212 and the collimating lens 220. For example, c_{offset} may be 30. In this example a light emitting diode 212 in the peripheral region that is 72 mm from the center of the array would have its Fresnel feature 230 shifted 2.4 mm inwards to 69.6 mm from the center of the array, but a light emitting diode 212 in the central region that is 9 mm from the center of the array would have its Fresnel feature 230 shifted 0.3 mm towards the center to a position of 8.7 mm.

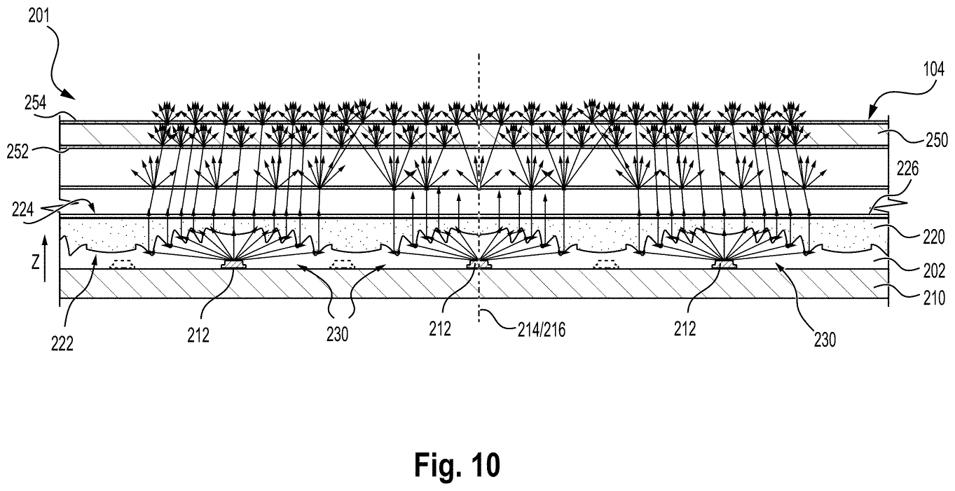

FIG. 10 shows an alternative optical assembly 201. The discussion of the optical assembly 200 herein also applies to this alternative optical assembly 201, and the same reference numerals are used to refer to the same or similar components or features. A detailed discussion of such features is omitted here, and the following discussion pertains to differences between the optical assembly 200 and the optical assembly 201. Instead of, or in addition to, offsetting the center axis of the Fresnel feature 230 from the corresponding light emitting diode 212, another Fresnel feature, referred to herein as an outer Fresnel feature 226, may be formed on the outer surface 224 of the collimating lens 220. The outer Fresnel feature 226 may be used to direct the rays of light emitted from those light emitting diodes 212 positioned further away from the longitudinal centerline 214 or the transverse centerline 216. The rays of light from these light emitting diodes 212 are directed toward the longitudinal centerline 214 and/or the transverse centerline 216, instead of parallel to the outward direction, causing the rays from the different light emitting diodes 212 to overlap on the target plane corresponding to the eyes 12 of a user 10. The outer Fresnel feature 226 may be centered with respect to the array of light emitting diodes 212, which in this embodiment is also the center point of the front surface 104 and the entire light dosage administering device 100. This outer Fresnel feature 226 blends the light from the different light emitting diodes 212 to establish a more uniform light distribution. In the embodiment shown in FIG. 10, the outer Fresnel feature 226 is molded on the outer surface 224 of the collimating lens 220, but this feature may also have other suitable forms, such as being a separate, thin lens, for example.

In the embodiments depicted in the figures, the LED board 210 is planar, and the outer surface 218 of the LED board 210 is a planar surface. Alternatively, however, the LED board 210, and more specifically the outer surface 218, may have a curved shape or profile. The curved shaped may be concave relative to one or both of the longitudinal direction and the transverse direction. With a curved shape, such as a concave shape, of the outer surface 218 the collimating lens 220 may also have a concave shape corresponding to the concave shape of the outer surface 218. The concave shape of the outer surface 218 may have a similar effect as the outer Fresnel feature 226 and thus the outer Fresnel feature 226 could be omitted in such an embodiment.

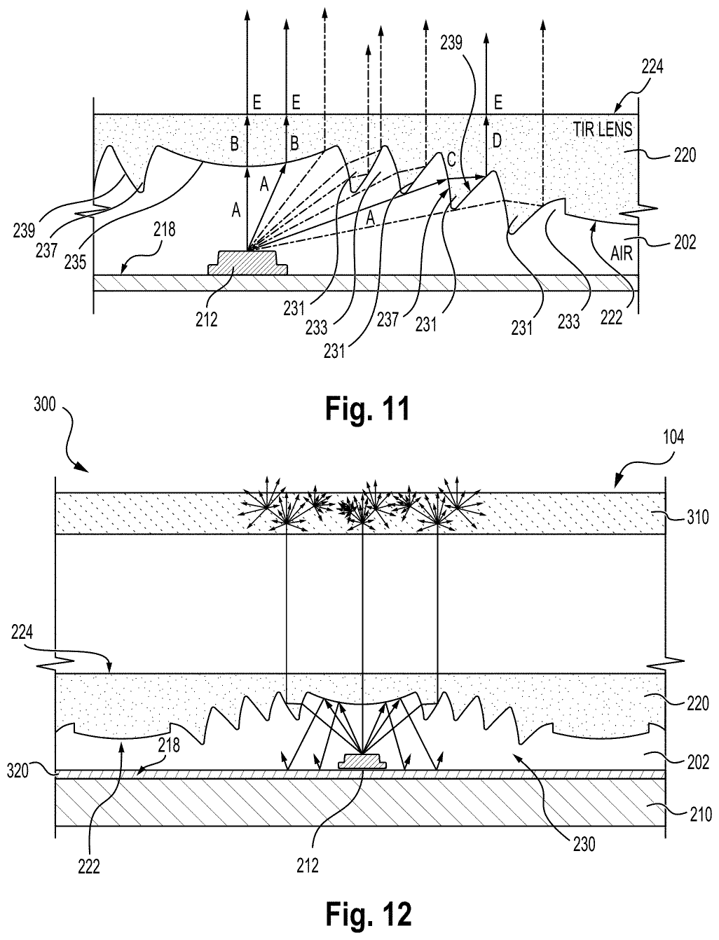

FIG. 11 is a detail view of a light emitting diode 212 and the collimating lens 220 in one embodiment. The collimating lens 220 of this embodiment is a total internal reflection (TIR) lens. Using a TIR lens further increases the efficiency of the optical assembly 200, as the light entering the collimating lens 220 is efficiently directed outward instead of having reflected rays that are directed inward back towards the LED board 210. FIG. 11 further illustrates the collimation of the rays using the Fresnel feature 230 on the inner surface 222 of the collimating lens 220. A gap 202 is formed between an outer surface 218 of the LED board 210 and the inner surface 222 of the collimating lens 220. The gap 202 in this embodiment contains air and the refractive index of the air is less than the refractive index of the collimating lens 220. Rays of light, labeled A, are emitted from the light emitting diode 212. An idealized point source is shown here for simplicity of description. Each Fresnel feature 230 includes a central dome 235, and some of the emitted rays entering the central dome 235 are refracted to align with, or be parallel to the central axis of the Fresnel feature 230. The rays refracted by the central dome 235 are labeled B. As noted above, the Fresnel feature 230 includes a plurality of concentric prisms 231. The concentric prisms 231 can also be referred to as teeth. Some of the emitted rays enter the front face 237 of the concentric prisms 231 and are refracted, rays labeled C, towards the back face 239 of the concentric prisms 231. When the rays labeled C contact the back face 239, they are below the critical angle of the interface between the Fresnel feature 230 and the gap 202 and then reflect, by total internal reflection, in a direction to be parallel to the central axis of the Fresnel feature 230. These reflected rays are labeled D. The rays labeled B and D are thus columnated and exit the outer surface 224 of the collimating lens 220 as rays labeled E.

Whether or not the collimating lens 220 is a TIR lens, an outer surface 218 (see FIGS. 5 and 8) of the LED board 210 may be reflective, such as having white color. A reflective outer surface 218 of the LED board 210 is more important to increase the efficiency when a TIR lens is not used as the collimating lens 220, as will be discussed further with reference to FIG. 12 below.

While the collimating lens 220 described above is based on Fresnel features, such configuration is simply one option, and it will be appreciated that the collimating lens 220 may be alternatively or additionally formed of other and/or additional optical features to provide collimating functions.

The collimating lens 220 may be formed from any suitable highly optically transmissive material that allows accurate ray tracing through the complex lens geometry. Such a material may be an injection moldable plastic. A suitable plastic includes, for example, Polymethyl methacrylate (PMMA). Injection molding these lenses allows for the complex geometry of the collimating lens 220 to be formed efficiently with a large volume of production.

After being collimated, the rays of light may still have non-uniform spatial distribution over, for example, the front surface 104. In one embodiment, the optical assembly 200 may include at least one diffusion layer to blend the light emitted from the light emitting diodes 212 and create a more uniform distribution of the light. Where light emitting diodes 212 having different wavelengths of light are used, the at least one diffusion layer may further establish a uniform color on the front surface 104. In the embodiment shown in FIGS. 6 and 7, the optical assembly 200 includes a plurality of diffusion layers, more specifically in this embodiment, a first diffusion layer, a second diffusion layer, and a third diffusion layer. The diffusion layers may be formed by different optical diffusers. The optical assembly 200 thus preferably includes at least one optical diffuser positioned outward of the collimating lens 220 in the eye facing direction and, more preferably, a plurality of optical diffusers positioned outward of the collimating lens 220 in the eye facing direction.

In one embodiment shown in FIGS. 6 and 7, the first diffusion layer is a diffusion film 240. The diffusion film 240 may also be referred to as a scattering film. In this embodiment, the diffusion film 240 is sandwiched between the collimating lens 220 and the transparent cover 250, but not optically adhered to either the collimating lens 220 or the transparent cover 250. In other embodiments a larger uniform air gap could be produced between the layers, using for example spacers. As schematically shown in FIGS. 6 and 7, the collimated rays of light are scattered or diffused when they strike the diffusion film 240. A diffusion film 240 may be desirable in this application for producing the desired diffusion effect while being thin and lightweight. Various suitable diffusion films 240 may be used including, but not limited to, textured films or prismatic scattering films. The diffusion film 240 preferably has a high transmissivity, but also good diffusivity, such as, for example, a diffusion film 240 having a transmissibility greater than 89% and a haze from 50% to 75%. A suitable diffusion film 240 may include, for example, PBS689G manufactured by Keiwa Incorporated. Instead of a single diffusion film 240, a plurality of diffusion films 240 may be used. FIG. 4 shows such an example. Alternatively, when the outer Fresnel feature 226 is not formed on the outer surface 224 of the collimating lens 220, this first diffusion layer may be texturing formed on the outer surface 224 of the collimating lens 220.

In one embodiment shown in FIGS. 6 and 7, the second and third diffusion layers are formed on surfaces of the transparent cover 250. In one embodiment, the transparent cover 250 is formed of glass to create a durable, but optically-clear, outer surface. The transparent cover 250 may be formed from Gorilla® Glass, such as Gorilla® Glass 6, manufactured by Corning Incorporated. In this embodiment, the volume of the transparent cover 250 itself is optically clear; but in other embodiments, the transparent cover 250 may also include features for volumetric or prismatic scattering. The transparent cover 250 includes an inner surface 252 and an outer surface 254. In one embodiment, the outer surface 254 of the transparent cover 250 constitutes the front surface 104 in this embodiment. The second diffusion layer is a haze applied to the inner surface 252 of the transparent cover 250, and the third diffusion layer is a haze applied to the outer surface 254 of the transparent cover 250. Any suitable method may be used to create the haze on the inner surface 252 and the outer surface 254 of the transparent cover 250, but in this embodiment, the haze is created by etching the transparent cover 250. In some embodiments, the haze on each of the inner surface 252 and outer surface 254 may be less than 60%.

In FIGS. 6 and 7 and moving outward from the light emitting diodes 212, the rays first contact the diffusion film 240 before contacting the inner surface 252 and the outer surface 254 of the transparent cover 250. From an optical diffusion standpoint, these layers could be reordered, and the diffusion film 240 may be located outward in the eye facing direction relative to the transparent cover 250. Such a construction, however, loses the protective benefits of the transparent cover 250 for the light dosage administering device 100, and more specifically, the optical assembly 200, and is thus not preferred. Likewise, for the optical assembly 201 shown in FIG. 10, the outer Fresnel feature 226 could be located in other locations within the optical assembly 201, such as outward in the eye facing direction relative to the diffusion film 240 or even the transparent cover 250.

Using at least three diffusion layers has been found to create a quality of the light that is both effective at treating the medical indications discussed above and creating a pleasing experience for the user 10. The light emitted from the front surface 104 of the light dosage administering device 100 may have a more uniform appearance rather than being perceived as discrete points of light, and the color may smoothly blend or transition from one color to the next for adjacent light emitting diodes 212 of different wavelengths. Other optical assemblies, however, may be used with the light dosage administering device 100 discussed herein. FIG. 12 is a schematic, cross-sectional view of an optical assembly 200 for a single light emitting diode 212 (light source) of an alternative optical assembly 300 that may be used with the light dosage administering device 100 discussed herein. The same reference numerals are used in FIG. 12 to refer to the same or similar components to those discussed above, and a detailed description of such components is omitted here. In the optical assembly 300 shown in FIG. 12, the optically clear transparent cover 250 has been replaced by a cover 310. The cover 310 of this embodiment is a cloudy medium that provides volumetric scattering of the collimated rays of light. The cover 310 may be a plastic such as high-density polyethylene (HDPE).

Although shown with only one diffusion layer (the cover 310), additional diffusion layers, such as the diffusion film 240 may be used in the optical assembly 300 shown in FIG. 12. In addition, other features discussed above may also be used in this embodiment, such as the outer Fresnel feature 226. Although a TIR lens may be used as the collimating lens 220 in this embodiment, the collimating lens 220 shown in FIG. 12 illustrates the collimating lens 220 reflecting some rays back towards the LED board 210; and, a reflector 320 is formed on the outer surface 218 of the LED board 210 to reflect these rays back outward in the eye facing direction. As noted above, the reflector 320 may include a reflective surface, such as a white color of the outer surface 218 of the LED board 210.

As noted above, the light dosage administering device 100 is used to administer a dosage of light and thus may be used in a method of administering a dosage of light. In such a method, the light dosage administering device 100 is placed or positioned in front of the eyes 12 of a patient (user 10). The light dosage administering device 100 may be positioned as discussed above. Light rays are then emitted from a light source, such as the array of light emitting diodes 212, as discussed above. In the embodiments of the light dosage administering device 100, the light rays are collimated and/or diffused, as discussed above, before being projected onto a target area. As discussed above, the target area includes the eyes 12 of the patient (user 10), and the collimated and/or diffused light rays are projected onto the target area while the eyes 12 of the patient (user 10) are closed.

Further aspects of the present disclosure are provided by the subject matter of the following clauses.

A light administering device including a light source configured to emit light rays and a collimating lens. The collimating lens is configured to collimate the light rays emitted from the light source such that the light rays are aligned in a light-emitting direction. The light administering device is configured to administer a dosage of light in the light-emitting direction towards the eyes of a user.

A light administering device capable of administering a dosage of light in a light-emitting direction towards the eyes of a user. The light administering device includes a plurality of light sources and a collimating lens. The plurality of light sources is arranged in an array of rows and columns. The array of light sources has at least three columns of light sources and at least three rows of light sources and more columns of light sources than rows of light sources. Each light source of the plurality of light sources is configured to emit light rays. The collimating lens is configured to collimate the emitted light rays from each light source of the plurality of light sources such that the light rays emitted from the plurality of light sources and collimated by the collimating lens are aligned in the light-emitting direction.

A light administering device capable of administering a dosage of light in a light-emitting direction towards the eyes of a user. The light administering device includes a light emitter and a collimating lens. The light emitter includes a plurality of light sources. The plurality of light sources includes a plurality of first light sources configured to emit light rays of a first wavelength, a plurality of second light sources configured to emit light rays of a second wavelength, and a plurality of third light sources configured to emit light rays of a third wavelength. The second wavelength is different from the first wavelength, and the third wavelength is different from the first wavelength and the second wavelength. The collimating lens is configured to collimate the emitted light rays from each light source of the light emitter such that light rays emitted from the light emitter and collimated by the collimating lens are aligned in the light-emitting direction.

A light administering device capable of administering a dosage of light in a light-emitting direction towards the eyes of a user. The light administering device includes a light source configured to emit light rays, a collimating lens, and at least one optical diffuser.

The collimating lens is configured to collimate the light rays emitted from the light source such that the light rays are aligned in the light-emitting direction. The at least one optical diffuser receives collimated light rays transmitted through the collimating lens and is configured to diffuse the collimated light rays.

The light administering device of any preceding clause, further configured to be placed from 5 cm to 40 cm in front of the eyes of the user.

The light administering device of any preceding clause, wherein the light source is a light emitting diode.

The light administering device of any preceding clause, wherein the collimating lens includes a Fresnel lens feature corresponding to the light source.

The light administering device of any preceding clause, wherein the Fresnel lens feature is provided on an outer surface of the collimating lens facing the light source.

The light administering device of any preceding clause, wherein the Fresnel lens feature is centered in alignment with the light source.

The light administering device of any preceding clause, wherein the collimating lens is a total internal reflection lens.

The light administering device of any preceding clause, further comprising at least one optical diffuser that receives the collimated light rays transmitted through the collimating lens and is configured to diffuse the collimated light rays.

The light administering device of any preceding clause, wherein one or more of the at least one optical diffuser is a diffusion film.

The light administering device of any preceding clause, wherein the diffusion film is a prismatic scattering film.

The light administering device of any preceding clause, further comprising a cover glass having a first outer surface facing the light source and a second outer surface facing the light-emitting direction. One or more of the at least one optical diffuser being a haze applied to at least one of the first outer surface and the second outer surface.

The light administering device of any preceding clause, wherein the haze is applied to each of the first outer surface of the cover glass and the second outer surface of the cover glass.

The light administering device of any preceding clause, wherein the haze applied to the first outer surface of the cover glass is different from the haze applied to the second outer surface of the cover glass.

The light administering device of any preceding clause, wherein each light source of the light emitter is a light emitting diode.

The light administering device of any preceding clause, wherein the collimating lens includes a plurality of Fresnel lens features, each light source of the light emitter corresponding to a respective Fresnel lens feature of the plurality of Fresnel lens features.

The light administering device of any preceding clause, wherein the Fresnel lens features are provided on an outer surface of the collimating lens facing the light emitter.

The light administering device of any preceding clause, wherein each respective Fresnel lens feature of the plurality of Fresnel lens features corresponding to a respective light source of the light emitter is centered in alignment with the respective light source.

The light administering device of any preceding clause, wherein the collimating lens is a total internal reflection lens.

The light administering device of any preceding clause, further comprising at least one optical diffuser that receives the collimated light rays transmitted through the collimating lens and is configured to diffuse the collimated light rays.

The light administering device of any preceding clause, wherein one or more of the at least one optical diffuser is a diffusion film.

The light administering device of any preceding clause, wherein the diffusion film is a prismatic scattering film.

The light administering device of any preceding clause, further comprising a cover glass having a first outer surface facing the light emitter and a second outer surface facing the light-emitting direction, one or more of the at least one optical diffuser being a haze applied to at least one of the first outer surface and the second outer surface.

The light administering device of any preceding clause, wherein the haze is applied to each of the first outer surface of the cover glass and the second outer surface of the cover glass.

The light administering device of any preceding clause, wherein the haze applied to the first outer surface of the cover glass is different from the haze applied to the second outer surface of the cover glass.

The light administering device of any preceding clause, wherein the plurality of light sources is arranged in a planar profile.

The light administering device of any preceding clause, wherein the plurality of light sources is arranged in a curved profile.

The light administering device of any preceding clause, wherein the plurality of light sources is provided on a reflective surface.

The light administering device of any preceding clause, wherein the array includes a centerline and the light administering device further comprises a Fresnel feature that receives collimated light transmitted through the collimated lens and is configured to direct collimated light rays located away from the centerline, towards the centerline.

The light administering device of any preceding clause, wherein the centerline is a longitudinal centerline extending in a row direction.

The light administering device of any preceding clause, wherein the centerline is a transverse centerline extending in a column direction.

The light administering device of any preceding clause, wherein at least two adjacent columns of light sources among the columns in the array are staggered with respect to one another.

The light administering device of any preceding clause, wherein each light source is a light emitting diode.

The light administering device of any preceding clause, wherein the collimating lens includes a plurality of Fresnel lens features, each light source corresponding to a respective Fresnel lens feature of the plurality of Fresnel lens features.

The light administering device of any preceding clause, wherein the Fresnel lens features are provided on an outer surface of the collimating lens facing the plurality of light sources.

The light administering device of any preceding clause, wherein each respective Fresnel lens feature of the plurality of Fresnel lens features corresponding to a respective light source is centered in alignment with the respective light source.

The light administering device of any preceding clause, wherein the collimating lens is a total internal reflection lens.

The light administering device of any preceding clause, further comprising at least one optical diffuser that receives the collimated light rays transmitted through the collimating lens and is configured to diffuse the collimated light rays.

The light administering device of any preceding clause, wherein one or more of the at least one optical diffuser is a diffusion film.

The light administering device of any preceding clause, wherein the diffusion film is a prismatic scattering film.

The light administering device of any preceding clause, further comprising a cover glass having a first outer surface facing the plurality of light sources and a second outer surface facing the light-emitting direction, one or more of the at least one optical diffuser being a haze applied to at least one of the first outer surface and the second outer surface.

The light administering device of any preceding clause, wherein the haze is applied to each of the first outer surface of the cover glass and the second outer surface of the cover glass.

The light administering device of any preceding clause, wherein the haze applied to the first outer surface of the cover glass is different from the haze applied to the second outer surface of the cover glass.

The light administering device of any preceding clause, wherein the light source is a light emitting diode.

The light administering device of any preceding clause, wherein the collimating lens includes a Fresnel lens feature corresponding to the light source.

The light administering device of any preceding clause, wherein the Fresnel lens feature is provided on an outer surface of the collimating lens facing the light source.

The light administering device of any preceding clause, wherein the Fresnel lens feature is centered in alignment with the light source.

The light administering device of any preceding clause, wherein the collimating lens is a total internal reflection lens.

The light administering device of any preceding clause, wherein one or more of the at least one optical diffuser is a diffusion film.

The light administering device of any preceding clause, wherein the diffusion film is a prismatic scattering film.

The light administering device of any preceding clause, further comprising a cover including the at least one optical diffuser.

The light administering device of any preceding clause, wherein the cover is a cloudy medium that provides volumetric scattering of the collimated rays of light.

The light administering device of any preceding clause, wherein the cover is a cover glass having a first outer surface facing the light source and a second outer surface facing the light-emitting direction, at least one of one or more of the at least one optical diffuser being a haze applied to at least one of the first outer surface and the second outer surface.

A method of administering a dosage of light. The method includes placing a light administering device in front of the eyes of a patient, emitting light rays from at least one light source of the light administering device, collimating the light rays emitted from the least one light source such that the light rays are aligned in a light-emitting direction, and projecting the collimated light rays onto a target area. The light-emitting direction is a direction towards the eyes of a patient. Target area includes the eyes of the patient, and the collimated light rays are projected onto the target area while the eyes of the patient are closed.

The method of the preceding clause, wherein the light administering device is placed from 5 cm to 40 cm in front of the eyes of the patient.

The method of any preceding clause, wherein the at least one light source is a light emitting diode.

The method of any preceding clause, wherein the light rays are collimated using a collimating lens with a Fresnel lens feature corresponding to the light source.

The method of any preceding clause, wherein the Fresnel lens feature is provided on an outer surface of the collimating lens facing the light source.

The method of any preceding clause, wherein the Fresnel lens feature is centered in alignment with the light source.

The method of any preceding clause, further comprising diffusing the collimated light rays with at least one optical diffuser.

The method of any preceding clause, wherein one or more of the at least one optical diffuser is a diffusion film.

The method of any preceding clause, wherein the diffusion film is a prismatic scattering film.

The method of any preceding clause, wherein the light administering device includes a cover glass having a first outer surface facing the at least one light source and a second outer surface facing the light-emitting direction, one or more of the at least one optical diffuser being a haze applied to at least one of the first outer surface and the second outer surface.

The method of any preceding clause, wherein the haze is applied to each of the first outer surface of the cover glass and the second outer surface of the cover glass.

The method of any preceding clause, wherein the haze applied to the first outer surface of the cover glass is different from the haze applied to the second outer surface of the cover glass.

The method of any preceding clause, wherein emitting light rays includes emitting light rays from a plurality of light sources. The plurality of light sources includes a plurality of first light sources emitting light rays of a first wavelength, a plurality of second light sources emitting light rays of a second wavelength, and a plurality of third light sources emitting light rays of a third wavelength. The second wavelength is different from the first wavelength, and the third wavelength is different from the first wavelength and the second wavelength. Collimating the light rays includes collimating the emitted light rays from each light source.

The method of any preceding clause, wherein the plurality of light sources is arranged in an array of rows and columns. The array of light sources has at least three columns of light sources and at least three rows of light sources and more columns of light sources than rows of light sources.

Although this invention has been described with respect to certain specific exemplary embodiments, many additional modifications and variations will be apparent to those skilled in the art in light of this disclosure. It is, therefore, to be understood that this invention may be practiced otherwise than as specifically described. Thus, the exemplary embodiments of the invention should be considered in all respects to be illustrative and not restrictive, and the scope of the invention to be determined by any claims supportable by this application and the equivalents thereof, rather than by the foregoing description.

1. A light administering device, comprising:
a light source configured to emit light rays; and
a collimating lens configured to collimate the light rays emitted from the light source such that the light rays are aligned in a light-emitting direction,
wherein the light administering device is configured to administer a dosage of light in the light-emitting direction towards the eyes of a user.

2. The light administering device of claim 1, further configured to be placed from 5 cm to 40 cm in front of the eyes of the user.

3. The light administering device of claim 1, wherein the light source is a light emitting diode.

4. The light administering device of claim 1, wherein the collimating lens includes a Fresnel lens feature corresponding to the light source.

5. The light administering device of claim 4, wherein the Fresnel lens feature is provided on an outer surface of the collimating lens facing the light source.

6. The light administering device of claim 4, wherein the Fresnel lens feature is centered in alignment with the light source.

7. The light administering device of claim 1, wherein the collimating lens is a total internal reflection lens.

8. The light administering device of claim 1, further comprising at least one optical diffuser that receives the collimated light rays transmitted through the collimating lens and is configured to diffuse the collimated light rays.

9. The light administering device of claim 8, wherein one or more of the at least one optical diffuser is a diffusion film.

10. The light administering device of claim 9, wherein the diffusion film is a prismatic scattering film.

11. The light administering device of claim 8, further comprising a cover glass having a first outer surface facing the light source and a second outer surface facing the light-emitting direction, one or more of the at least one optical diffuser being a haze applied to at least one of the first outer surface and the second outer surface.

12. The light administering device of claim 11, wherein the haze is applied to each of the first outer surface of the cover glass and the second outer surface of the cover glass.

13. The light administering device of claim 12, wherein the haze applied to the first outer surface of the cover glass is different from the haze applied to the second outer surface of the cover glass.

14.-27. (canceled)

28. A light administering device capable of administering a dosage of light in a light-emitting direction towards the eyes of a user, the light administering device comprising:
a light emitter including a plurality of light sources, the plurality of light sources including:
a plurality of first light sources configured to emit light rays of a first wavelength,
a plurality of second light sources configured to emit light rays of a second wavelength, the second wavelength being different from the first wavelength, and
a plurality of third light sources configured to emit light rays of a third wavelength, the third wavelength being different from the first wavelength and the second wavelength; and
a collimating lens configured to collimate the emitted light rays from each light source of the light emitter such that light rays emitted from the light emitter and collimated by the collimating lens are aligned in the light-emitting direction.

29. The light administering device of claim 28, wherein each light source of the light emitter is a light emitting diode.

30. The light administering device of claim 28, wherein the collimating lens includes a plurality of Fresnel lens features, each light source of the light emitter corresponding to a respective Fresnel lens feature of the plurality of Fresnel lens features.

31.-33. (canceled)

34. The light administering device of claim 28, further comprising at least one optical diffuser that receives the collimated light rays transmitted through the collimating lens and is configured to diffuse the collimated light rays.

35.-39. (canceled)

40. The light administering device of claim 1, wherein the light source is one light source of a plurality of light sources arranged in an array of rows and columns, the array of light sources having at least three columns of light sources and at least three rows of light sources and more columns of light sources than rows of light sources, each light source of the plurality of light sources being configured to emit light rays, and
wherein the collimating lens configured to collimate the emitted light rays from each light source of the plurality of light sources such that the light rays emitted from the plurality of light sources and collimated by the collimating lens are aligned in the light-emitting direction.

41. The light administering device of claim 40, wherein the plurality of light sources is arranged in one of a planar profile or a curved profile.

42. (canceled)

43. The light administering device of claim 40, wherein the plurality of light sources is provided on a reflective surface.

44. The light administering device of claim 40, wherein the array includes a centerline and the light administering device further comprises a Fresnel feature that receives collimated light transmitted through the collimated lens and is configured to direct collimated light rays located away from the centerline, towards the centerline.

45. The light administering device of claim 44, wherein the centerline is one of a longitudinal centerline extending in a row direction or a transverse centerline extending in a column direction.

46. (canceled)

47. The light administering device of claim 40, wherein at least two adjacent columns of light sources among the columns in the array are staggered with respect to one another.

48.-58. (canceled)

59. A light administering device capable of administering a dosage of light in a light-emitting direction towards the eyes of a user, the light administering device comprising:
a light source configured to emit light rays;
a collimating lens configured to collimate the light rays emitted from the light source such that the light rays are aligned in the light-emitting direction; and
at least one optical diffuser receiving collimated light rays transmitted through the collimating lens and configured to diffuse the collimated light rays.

60. The light administering device of claim 59, wherein the light source is a light emitting diode.

61. The light administering device of claim 59, wherein the collimating lens includes a Fresnel lens feature corresponding to the light source.

62.-64. (canceled)

65. The light administering device of claim 59, wherein one or more of the at least one optical diffuser is a diffusion film.

66. (canceled)

67. The light administering device of claim 59, further comprising a cover including the at least one optical diffuser.

68. The light administering device of claim 67, wherein the cover is a cloudy medium that provides volumetric scattering of the collimated rays of light.

69. The light administering device of claim 67, wherein the cover is a cover glass having a first outer surface facing the light source and a second outer surface facing the light-emitting direction, at least one of one or more of the at least one optical diffuser being a haze applied to at least one of the first outer surface and the second outer surface.
